(19) United States
(12) Patent Application Publication
(10) Pub. No.: US 2026/0232869 A1
(43) Pub. Date: Aug. 13, 2026

(54) COMPOSITIONS AND METHODS FOR THREE-DIMENSIONAL (3D) BIOPRINTING

(71) Applicant: The Regents of the University of California, Oakland, CA (US)

(72) Inventors: Rahim Esfandyarpour, Irvine, CA (US); Ozdal Boyraz, Irvine, CA (US); Jorge Tavares-Negrete, Irvine, CA (US); Ceren Babayigit, Irvine, CA (US)

(21) Appl. No.: 19/152,668

(22) PCT Filed: Mar. 29, 2024
(86) PCT No.: PCT/US2024/022236

§ 371 Date: Jul. 31, 2025

Related U.S. Application Data
(60) Provisional application No. 63/492,950, filed on Mar. 29, 2023.

(51) Int. Cl. A61L 27/52; A61L 27/26; A61L 27/38; B33Y 10/00

(52) U.S. Cl. CPC A61L 27/52; A61L 27/26; A61L 27/3891; B33Y 10/00

(57) ABSTRACT

As a three-dimensional (3D) bioprinting technique, digital light processing (DLP) has become popular due to its capability to provide high-throughput and high-resolution constructs with precise chemical and biological factor distributions. However, despite the advancements in DLP technology, several hurdles remain, including phototoxicity, extensive printing time, and the limited portfolio of biocompatible/photo-cross-Jinkable materials. Herein, a novel DLP-based 3D-bioprinting technology′ called photopolymerization of orderly-extruded multi-materials (POEM) has been developed and fully characterized. The utility of the POEM technique for rapid and high-resolution 3D-printing of multi-material, multi-layer, and cell-laden structures is demonstrated. The printed configurations showed high cell viability (−80%) and metabolic activity′ for more than five days. The light-based POEM technique described herein enables the fabrication of 3D-cell-laden structures in a multi-material and multi-layer printing manner in biocompatible/photo-cross-linkable materials essential to construct complex heterogeneous tissues/organs.

### CROSS-REFERENCES TO RELATED APPLICATIONS

This application claims benefit of U.S. Provisional Application No. 63/492,950 filed Mar. 29, 2023, the specification of which is incorporated herein in its entirety by reference.

### FIELD OF THE INVENTION

The present invention features compositions and methods for three-dimensional (3D) bioprinting.

### BACKGROUND OF THE INVENTION

Bioprinting technology has rapidly advanced to provide a powerful platform to build anatomical and physiological relevant constructs. Previously, for more than the last 70 years, traditional two-dimensional (2D) approaches have been used in biomedical research (i.e., cell monolayers). However, these models have discrepancies, such as tissue heterogeneity and the poor recapitulation of human physiology, compared to an in vivo three-dimensional (3D) architecture (e.g., organs, tissues, tissue interaction, vascularization, and cell communication). On the other hand, 3D-bioprinting offers unique spatial control over the 3D architecture of the biological components by combining hydrogels, cells, and signaling molecules to recreate actual tissues. Moreover, 3D-bioprinting contributes significant advances in drug screening, disease modeling, high throughput assays, cancer research, biofabrication, and clinical translation. In these regards, 3D-bioprinting technologies have been widely developed in tissue engineering and biomedical applications.

The foremost 3D-bioprinting technologies have been advanced with nozzle- and light-based techniques. The most common technique of nozzle-based bioprinting is extrusion bioprinting. The vat-photopolymerization techniques such as stereolithography (SLA/SL), two-photon polymerization (TPP), and digital light processing (DLP) are categorized as the most frequently used light-based 3D-bioprinting technologies. Compared to the nozzle-based techniques, the light-based techniques provide more improved bioprinting resolution with smoother surfaces, fast printing speed, and high cell viability. Among the light-based technologies, the DLP has been more attention to 3D-bioprinting as a high-throughput technique with the capability to create high-resolution constructs with precise distributions of chemical and biological factors. However, DLP bioprinting has some hurdles, including irradiating photons from the light source that leads to phototoxicity and DNA mutations. The extensive printing times stress the cells and increase the chances of contamination. Moreover, it has a limitation in the portfolio of photo-cross-linkable and biocompatible materials.

Recently, two DLP volumetric additive manufacturing techniques, including Xolography and computed axial lithography (CAL), have attracted significant attention in light-based 3D-bioprinting technology. Both of these DLP techniques bear on the imaging processing part that requires complex operations, including skilled personnel and strong computational power. Nevertheless, the resins used in those works lack biocompatibility, precluding their translation to health applications. Furthermore, the current configuration of the resin reservoirs makes the volumetric printing of multi-materials impossible. For these reasons, the patterning of cell-laden polymers has remained a challenge to be addressed in terms of material selection, light intensity, and printing conditions. Hence, a biocompatible, multi-material, and rapid 3D-bioprinting system is yet to be developed.

Described herein the present invention addresses all the aforementioned limitations and presents a photopolymerization of orderly extruded multi-materials (POEM), a novel 3D-bioprinting technique, to print 3D multi-layer, multi-material, and cell-laden tissue structures with high cell viability and good resolution. The technique relies on the implementation of biocompatible, photo-cross-linkable, and customizable hydrogels for rapid printing of multi-material and complex physiological relevant in vivo mimicking structures. The POEM technique principle of working is extrusion bioprinting of photo-cross-linkable hydrogels in a layer-by-layer manner followed by high-resolution patterning of the layers to the desired shapes and configurations using a 4-f lens system. The POEM technique described herein maintains a high printing resolution, offers multi-material printing to achieve the heterogeneity presented in actual tissues and multicellular structures, eliminates cross-contamination and the cleaning process that is required in other state-of-the-art multi-material printing techniques, and enables multilayer printing to structurally mimic the in vivo tissue architectures. Moreover, the use of a support bath implemented in the POEM technique holds the 3D-bioprinted structures still during the entire process and hence eliminates the chance of collision and any structural deformations. Elevated viscosity within the support bath acts as a barrier to the diffusion of free radicals and monomers, resulting in a reduction of scattering-induced photopolymerization in unintended regions. This phenomenon effectively enhances the resolution of the system.

### BRIEF SUMMARY OF THE INVENTION

It is an objective of the present invention to provide compositions and methods that allow for multi-layer and multi-material printing of biocompatible and/or photo-cross-linkable materials to fabricate physiologically relevant cell-laden structures, as specified in the independent claims. Embodiments of the invention are given in the dependent claims. Embodiments of the present invention can be freely combined with each other if they are not mutually exclusive.

Described herein is a novel DLP-based 3D-bioprinting technology called photopolymerization of orderly extruded multi-materials (e.g., POEM) that has been developed and fully characterized. The POEM technique principle of working is extrusion bioprinting of photocrosslinkable hydrogels in a layer-by-layer manner followed by high-resolution patterning of the layers to the desired shapes and configurations using a 4-f lens system. The utility of the POEM technique for rapid and high-resolution 3D printing of multi-material, multi-layer, and cell-laden structures is demonstrated. The printed configurations showed high cell viability (>80%) and metabolic activity. The POEM technique here maintains a high printing resolution, offers multi-material printing to achieve the heterogeneity presented in actual tissues and multicellular structures, eliminates cross-contamination and the cleaning process that is required in other state-of-the-art multi-material printing techniques, and enables multi-layer printing to structurally mimic the in-vivo tissue architectures.

Moreover, the use of a support bath implemented in the POEM technique holds the 3D-bioprinted structures still during the entire process and hence eliminates the chance of collision and any structural deformations. Based on the POEM technique, sample 3D architectures (i.e., hexagonal ring, wheel, and cylinder structures) were successfully printed with a high resolution (65 μm) and analyzed by fluorescent imaging, indicating that the POEM enables printing along the x-, y-, and z-axis. The geometries were mimicked from the structure of the hepatic hexagonal lobule units, the hepatic interconnection lobule-vasculature, and the esophagus multi-layered heterogeneity. Physiologically relevant esophagus models were bio-fabricated in a hollow tubular scale-down fashion to verify the POEM technique. To achieve biocompatibility, biomaterial formulations are used that are tailored according to the needs of cells to increase the survival rate and to obtain non-toxic resins as opposed to previous works. The methods described herein provide a novel 3D-bioprinting technique for the field of bioprinting multi-material and complex architectures mimicking in-vivo 3D tissues with high resolution.

In some embodiments, the present invention features a method for three-dimensional (3D) bioprinting. The method may comprise (a) preparing one or more prepolymers and a plurality of cells incorporated into at least one of the one or more prepolymers and (b) patterning the one or more prepolymers by crosslinking the one or more prepolymers to form a hydrogel. In some embodiments, the plurality of cells are encapsulated in the hydrogel.

In some embodiments, the present invention features a three-dimensional (3D) printed article comprising a hydrogel and cells.

In some embodiments, the present invention may also feature a three-dimensional (3D) printer cartridge containing a printing solution comprising a prepolymer, a plurality of cells, and a photo-initiator.

One of the unique and inventive technical features of the present invention is the development of a 3D-bioprinting technique to print 3D multi-layer, multi-material, and cell-laden tissue structures with high cell viability and good resolution. Without wishing to limit the invention to any theory or mechanism, it is believed that the technical feature of the present invention advantageously provides for rapid printing of multi-material and complex physiological relevant in vivo mimicking structures. The methods described herein allow for the extrusion bioprinting of photo-cross-linkable hydrogels in a layer-by-layer manner, followed by high-resolution patterning of the layers to the desired shapes and configurations using a 4-f lens system. This maintains a high printing resolution, offers multi-material printing to achieve the heterogeneity presented in actual tissues and multicellular structures, eliminates cross-contamination and the cleaning process that is required in other state-of-the-art multi-material printing techniques, and enables multi-layer printing to structurally mimic the in-vivo tissue architectures. None of the presently known prior references or work has the unique, inventive technical feature of the present invention.

Furthermore, the prior references teach away from the present invention. For example, state-of-the-art techniques bear on complex imaging processing, require highly skilled personnel, and operate with non-biocompatible/photo-cross-linkable materials. Additionally, they are not yet capable of multi-layer and multi-material printing of biocompatible/photo-cross-linkable materials to fabricate physiologically relevant cell-laden structures.

Furthermore, the inventive technical features of the present invention contributed to a surprising result. One of ordinary skill in the art would find it impossible to pattern prepolymers incorporating living cells because photo-cross-linkable prepolymers with cell viability tend to mix, thus nullifying the patterning. Surprisingly, the implementation of a thickening agent, such as Carbopol-940, temporarily increases the viscosity of the prepolymers, which allows for patterning to be maintained while also maintaining the cell viability of the prepolymers. Thus, the inventive technical feature of the present invention contributed to a surprising result.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show a schematic of the POEM technique printing configuration. FIG. 1A shows a multilayer vat that was prepared with extrusion printing as longitudinal or lateral layering configurations. The photographic view of the printed longitudinal layering case was presented with a coin to show the comparison of structural dimensions. FIG. 1B shows the prepared vat was placed in the DLP printing system and exposed to patterned UV light (λ=395 nm). FIG. 1C shows after the crosslinking process, printed objects were removed from the vat and washed with PBS to clean the support bath on the printed object.

FIGS. 2A, 2B, 2C, 2D, 2E, 2F, 2G, 2H, and 2I show the application of the POEM technique for the fabrication of multi-material structures. FIG. 2A shows a digital mask for a hexagonal ring pattern. FIG. 2B shows a top view, and FIG. 2C shows a side view of the DLP printed pattern for the hexagonal ring. The enlarged microscope image of the printed pattern was given as an inset in FIG. 2B. FIG. 2D shows a digital mask for the wheel pattern. FIG. 2E shows a top view, and FIG. 2F shows a side view of the DLP printed pattern for the wheel. The enlarged microscope image of the printed pattern was given as an inset in FIG. 2E. FIG. 2G shows a top view of a DLP-printed cylinder with a longitudinal layering arrangement with three colors. FIG. 2H shows a side view of a DLP-printed cylinder with a lateral layering arrangement. FIG. 2I shows z-stack imaging of the cylinder in a lateral layering configuration with two colors.

FIGS. 3A and 3B show 3D cell-laden bioprinting experiments. FIG. 3A shows a quantitative analysis of the cell viability assay. (N=3, n=4, ***p≤0.001) and FIG. 3B shows Resazurin assay on cells encapsulated in bioprinted constructs. (N=3).

FIG. 4A shows resolution analyses of the system concerning the exposure time.

FIG. 4B shows an example of the superposition of the masks at appropriate times to achieve high and low-resolution patterns in the same structure.

FIG. 4C shows a flow curve.

FIG. 4D shows an absorption spectrum.

FIG. 4E shows water swelling characteristics, and FIG. 4F shows the mechanical properties of the materials.

FIGS. 5A, 5B, and 5C show photoinduced reaction pathways of LAP and free-radical-induced cross-linking of PEGDA (FIG. 5B) and GelMA (FIG. 5C).

FIGS. 6A and 6B show a schematic representation of the prepared multi-layer reservoir using digital masks and expected printing output. FIG. 6B shows the superposition of the masks to obtain the same accuracy for multi-material printing.

FIGS. 7A, 7B, 7C, and 7D show a top and view of a DLP printed hemisphere with a single material (FIGS. 7A and 7B) and multi-material with two colors (FIG. 7C), longitudinal layering arrangement. FIG. 7D shows compression modulus characteristics of single and multi-material printed structures.

FIG. 8A shows a table of the dimensions of original designs and DLP printed patterns (n=5) (w1/w2/w3: width, s: side, h: height, D: diameter and θ1/θ2/θ3: corner angle)).

FIG. 8B shows a table of the dimensions of original designs and DLP-printed multi-material patterns (n=5).

### DETAILED DESCRIPTION OF THE INVENTION

The term “prepolymer” is defined herein as a monomer or system of monomers that have been reacted to an intermediate-molecular mass state.

The term “photo-cross-linking” is defined herein as a covalent cross-linking method by visible light irradiation of a photocatalyst in the presence of an electron acceptor and the protein of interest.

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Based on the POEM technique, sample 3D architectures (i.e., hexagonal ring, wheel, and cylinder structures) were successfully printed with a high resolution (65 μm) and analyzed by fluorescent imaging, indicating that the POEM enables printing along the x-, y-, and z-axis. The geometries were mimicked from the structure of the hepatic hexagonal lobule units, the interconnection hepatic lobule-vasculature, and the esophagus multi-layered heterogeneity. Physiologically relevant esophagus models were bio-fabricated in a hollow tubular scale-down fashion to verify the POEM technique. To achieve biocompatibility, biomaterial formulations that are tailored according to the needs of cells were used to increase the survival rate, and to obtain non-toxic resins as opposed to previous works. In this context, a blend of poly(ethylene glycol) diacrylate (PEGDA) and gelatin methacryloyl (GelMA) polymers was designed to be photo-patterned and provided scaffolding to the muscle (C2C12) and fibroblast cells (L929). The esophagus-like structures maintain up to 80% cell viability and are metabolically active after printing and during the time (~5 days), meaning that the POEM technique is suitable to be used in biomedical fields. Hence, the POEM technique paves the way for the 3D-bioprinting of multi-material and complex architectures closer to 3D-tissues with high resolution by using custom-made bioink formulations. With a custom selection of materials according to the application, it enables the printing of multi-material structures and the rapid bioprinting of cell-loaded structures with a simple post-processing routine.

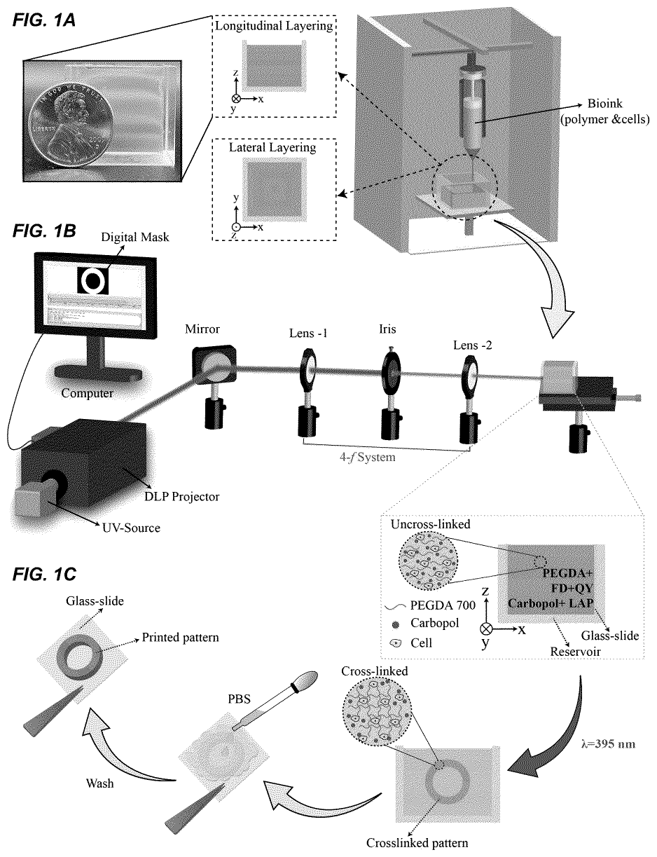

Referring now to FIGS. 1A-7D, the present invention features compositions and methods (e.g., POEM technique) that can perform rapid and high-resolution 3D bioprinting of multi-material, multi-layer, and cell-laden structures. The printed configurations can maintain high cell viability (>80%) and metabolic activity. The POEM technique maintains a high printing resolution, offers multi-material printing to achieve the heterogeneity presented in actual tissues and multicellular structures, eliminates cross-contamination and the cleaning process that is required in other state-of-the-art multi-material printing techniques, and enables multi-layer printing to structurally mimic the in-vivo tissue architectures. Moreover, the POEM technique holds the 3D-bioprinted structures still during the entire process and hence eliminates the chance of collision and any structural deformations. The proposed POEM technique is a novel 3D-bioprinting technique for the field of bioprinting multi-material and complex architectures mimicking in-vivo 3D tissues with high resolution.

The present invention features a method for three-dimensional (3D) bioprinting. The method may comprise (a) preparing one or more prepolymers and a plurality of cells incorporated into at least one of the one or more prepolymers and (b) patterning the one or more prepolymers by crosslinking the one or more prepolymers to form a hydrogel. In some embodiments, the plurality of cells are encapsulated in the hydrogel.

In some embodiments, the one or more prepolymers comprise poly(ethylene glycol) diacrylate (PEGDA), gelatin methacryloyl (GelMA), or a combination thereof.

Preparing the one or more prepolymers may comprise putting the one or more prepolymers into a printer cartridge and extruding the one or more prepolymers into a reservoir. In some embodiments, the one or more prepolymers are extruded into a specific pattern and/or structure, e.g., a hexagonal ring, a wheel, or a cylinder structure.

In some embodiments, the one or more prepolymers are extruded as one or more layers. In some embodiments, the one or more layers of prepolymer comprise distinct boundaries between the one or more layers of prepolymer.

In some embodiments, the plurality of cells comprise endothelial cells or fibroblast cells.

In some embodiments, a digital light processing (DLP)-printing system is used for patterning the one or more prepolymers. The DLP-print system may comprise (i) an ultraviolet (UV) light source; (ii) a digital micromirror device (DMD) chipset; (iii) a computer to adjust and send a digital mask sequence; (iv) an optical projection lens system; and (v) a stage for sample positioning.

The methods described herein may further comprise post-processing of the hydrogel. Post-processing of the hydrogel may comprise washing the hydrogel to remove the non-crosslinked prepolymer and dissolve the support bath (exp: Carbopol-940). In some embodiments, the hydrogel is washed with a phosphate-buffered saline (PBS) solution.

The present invention may also feature a three-dimensional (3D) printer cartridge containing a printing solution comprising a prepolymer, a plurality of cells, and a photo-initiator.

In some embodiments, the photo-initiator is a biocompatible photo-initiator. In some embodiments, the photo-initiator is lithium phenyl-2,4,6-trimethylbenzoylphosphinate (LAP).

In some embodiments, the cells comprise muscle cells or fibroblast cells. In some embodiments, the prepolymer comprises poly(ethylene glycol) diacrylate (PEGDA), gelatin methacryloyl (GelMA), or a combination thereof.

In some embodiments, the 3D cartridge may further comprise Carbopol-940.

The present invention may further feature a three-dimensional (3D) printed article comprising a hydrogel and cells. In some embodiments, the hydrogel is formed from a prepolymer, e.g., poly(ethylene glycol) diacrylate (PEGDA), gelatin methacryloyl (GelMA), or a combination thereof.

In some embodiments, the article is shaped into a hexagonal ring, a wheel, or a cylinder structure.

In some embodiments, the present invention features a method for three-dimensional (3D) bioprinting of multi-layer multi-material structures. In some embodiments, the method may comprise preparing a plurality of prepolymers and a plurality of living cells incorporated into at least one of the plurality of prepolymers. The plurality of prepolymers may comprise a plurality of prepolymer types. The plurality of living cells may comprise a plurality of cell types. The method may further comprise applying a thickening agent to the plurality of prepolymers. The method may further comprise patterning the plurality of prepolymers into a three-dimensional shape comprising one or more layers such that adjacent prepolymers differ in prepolymer type, cell type of one or more living cells incorporated into each prepolymer, or a combination thereof. Patterning may comprise photo-crosslinking the plurality of prepolymers to form a hydrogel. The plurality of living cells may be encapsulated in the hydrogel.

In some embodiments, the plurality of prepolymer types may comprise poly(ethylene glycol) diacrylate (PEGDA), gelatin methacryloyl (GelMA), Alginate Methacrylate (ALMA), Silk methacrylate (SilMA), collagen methacrylate (ColMA), methacrylated poly(vinyl alcohol) (PVA-MA), Methacryloyl-substituted recombinant human tropoelastin (MeTro), any decellularized ExtraCellular Matrix-methacrylate (dECM-MA), or a combination thereof. In some embodiments, preparing the plurality of prepolymers may comprise putting the plurality of prepolymers into a printer cartridge and extruding the plurality of prepolymers into a reservoir. In some embodiments, the plurality of prepolymers may be extruded into a hexagonal ring, a wheel, or a cylinder structure. In some embodiments, the one or more layers of prepolymer may comprise distinct boundaries between the one or more layers of prepolymer.

In some embodiments, a digital light processing (DLP)-printing system may be used for patterning the plurality of prepolymers. In some embodiments, the DLP-print system may comprise an ultraviolet (UV) light source configured to generate UV light. The system may further comprise a computing device comprising a processor configured to execute computer-readable instructions and a memory component operatively coupled to the processor, comprising computer-readable instructions for generating, adjusting, and sending a digital mask sequence. The system may further comprise a digital micromirror device (DMD) chipset communicatively coupled to the computing device, disposed optically in-line with the UV light source, configured to receive the digital mask sequence and modulate the UV light based on the digital mask sequence to form a digital mask image. The system may further comprise an optical projection lens system disposed optically in-line with the DMD chipset, configured to project the digital mask image. The system may further comprise a stage for sample positioning disposed optically in-line with the optical projection lens system. The digital mask image may be projected onto the stage.

In some embodiments, the optical projection lens system may be further configured to filter the digital mask image at a Fourier plane. Filtering the digital mask image at the Fourier plane may be used to increase the resolution and sharpness of the digital mask image. In some embodiments, the DMD chipset may comprise a plurality of mirrors. In some embodiments, the memory component may further comprise instructions for controlling an operation time of each mirror of the plurality of mirrors of the DMD chipset. In some embodiments, controlling the individual mirror operation times may control total light intensity at desired points on the digital mask image. In some embodiments, the method may further comprise post-processing of the hydrogel. In some embodiments, post-processing of the hydrogel may comprise washing the hydrogel to remove non-crosslinked prepolymer. In some embodiments, the hydrogel may be washed with a phosphate-buffered saline (PBS) solution.

The present invention features a multi-layer, multi-material, three-dimensional (3D) printed article comprising a hydrogel and living cells. The hydrogel may be formed from a pattern of prepolymers comprising a thickening agent. The living cells may be incorporated into the pattern of prepolymers. Adjacent prepolymers in the pattern of prepolymers differ in prepolymer type, cell type, or a combination thereof. In some embodiments, the article may be shaped into a hexagonal ring, a wheel, or a cylinder structure.

The present invention features a three-dimensional (3D) printer cartridge containing a printing solution comprising a plurality of prepolymers having a plurality of prepolymer types, a plurality of living cells having a plurality of cell types, a thickening agent applied to the plurality of prepolymers, and a photo-initiator. In some embodiments, the photo-initiator may be a biocompatible photo-initiator. In some embodiments, the photo-initiator may be lithium phenyl-2,4,6-trimethylbenzoylphosphinate (LAP), 2-Hydroxy-4′-(2-hydroxyethoxy)-2-methylpropiophenone (Irgacure 2959), Eosin Y, VA086, or a combination thereof. In some embodiments, the plurality of cell types may comprise endothelial cells, fibroblast cells, epithelial cells, muscle cells (myocytes), bone cells (osteocytes), cartilage cells (chondrocytes), heart muscle cells (cardiomyocytes), liver cells (hepatocytes), neurons, fat cells (adipocytes), keratinocytes, cancerous cells, stem cells (induced pluripotent stem cells, embryonic stem cells, mesenchymal stem cells, adipose-derived stem cells, amniotic fluid stem cells, myeloid-derived suppressor cells), macrophages, or a combination thereof. In some embodiments, the plurality of prepolymer types may comprise poly(ethylene glycol) diacrylate (PEGDA), gelatin methacryloyl (GelMA), Alginate Methacrylate (ALMA), Silk methacrylate (SilMA), collagen methacrylate (ColMA), methacrylated poly(vinyl alcohol) (PVA-MA), Methacryloyl-substituted recombinant human tropoelastin (MeTro), any decellularized ExtraCellular Matrix-methacrylate (dECM-MA), or a combination thereof. In some embodiments, the thickening agent may comprise Carbopol-940, Gelatin, Alginate, or a combination thereof.

In some embodiments, the computing device may be configured to couple to the DMD chipset by a wired connection. In some embodiments, the computing device may be configured to couple to the DMD chipset by a wireless connection. In some embodiments, the wireless connection may comprise a Bluetooth™ connection, a WiFi connection, a WLAN connection, a radiofrequency (RF) connection, or any other form of wireless connection.

In some embodiments, the three-dimensional structures of the present invention may be layered such that adjacent layers of prepolymer and cells are different in an X-axis, a Y-axis, a Z-axis, or a combination thereof. In some embodiments, the adjacent layers may differ in the types of prepolymer, the types of cells incorporated into the prepolymers, or a combination thereof. In some embodiments, the structure may comprise at most ten prepolymer types. In some embodiments, the structure may comprise at most ten cell types. In some embodiments, all prepolymers used in the three-dimensional structures may be cell-viable, photo-cross-linkable, photo-absorbent, or a combination thereof. In some embodiments, the ratio of cross-linking in the prepolymers may be controlled such that resolution loss in the 3D-printed model is prevented. If this was not used, the cross-linking reaction would go outside of the bounds defined by the input model. This allows for localized photopatterning and maintains the mechanical stability of the 3D model.

### EXAMPLE

The following is a non-limiting example of the present invention. It is to be understood that said example is not intended to limit the present invention in any way. Equivalents or substitutes are within the scope of the present invention.

The Design Approach and Principle of Operation: The POEM technique can be separated into three main categories as preparation of the multi-material vats, patterning with the DLP system, and cleaning of the printing object as post-process (FIGS. 1A, 1B, and 1C). In the multi-material vat preparation part, it was required to place the various prepolymers (i.e., PEGDA or PEGDA+GelMA) in printer cartridges and neatly extrude them into the quartz reservoir (FIG. 1A). Depending on the desired application, this extrusion process could be modeled in many configurations (e.g., longitudinal, lateral, etc.), applying different material thicknesses by tuning the printing speed, the needle gauge, and the G-code to the ink viscosity. The extrusion optimization will result in sharp boundaries between material layers. In contrast, the interconnection between multi-material layers resembles the anisotropic transition found in actual tissues. For example, the skin layers, bone-tendon junction, and gastrointestinal layers have intricate microscale anisotropic transitions. The complex hierarchical anisotropic topography directly impacts cell response by regulating their morphology, migration, and proliferation. To illustrate an optimized printing process, fluorescent particles with different colors were added to the prepolymer solutions. Then, they were extruded into longitudinal layers and concentric squares to generate lateral layering. In FIG. 1A, the photographic view was given for the longitudinal layering case with three colors. For the patterning part, the proposed DLP-printing system consisted of i) an ultraviolet (UV) light source (395 nm, Darkbeam); ii) a digital micromirror device (DMD) chipset; iii) a computer to adjust and send the digital mask sequence; iv) optical projection lens system; and v) a stage for sample positioning, as illustrated in FIG. 1B. In particular, the DMD chipset modulated the light by its 2D pixel arrays. From this modulation property, it has been deployed in many optical applications as a spatial light modulator to generate optical arbitrary waveforms, beam steerer for lidar applications, beam deflector for fast dispersive laser scanning systems. Here, the DMD chipset functioned as a photomask to generate dynamic optical patterns. The DMD configuration with tilted mirrors has often been modeled as a blazed grating. Hence, the reflected beam from DMD contains some diffraction orders. It was important to retrieve in-focus images to be able to obtain sharp images which were directed to the bioink for the patterning process. For this retrieving process, a 4-f imaging system was introduced in the system to increase the image quality by introducing filtering at the Fourier plane. Moreover, one can introduce a resolution versatile-projection ratio, by changing the focal length of the lenses in the 4-f system.

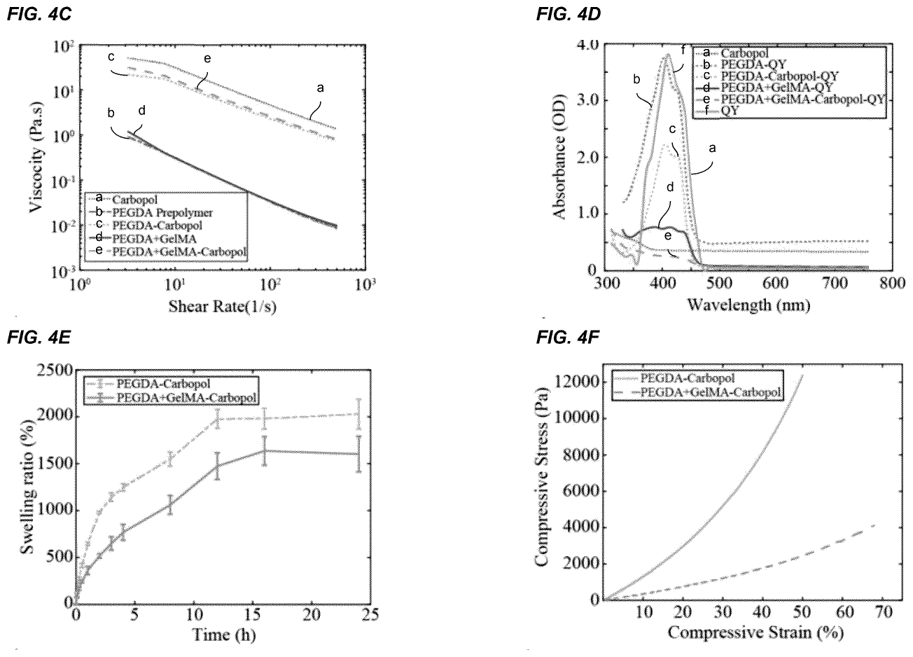

For the 3D-bioprinting process, glass slides were placed against the inner walls of a quartz reservoir to remove the pattern without damaging it (FIG. 1C). Then the reservoir was filled with prepolymer solution, which was composed of poly(ethylene glycol) diacrylate (PEGDA) 700 or a blend 2:1 of 20% PEGDA 700 and 10% gelatin methacryloyl (GelMA) as photo-cross-linkable polymers, Carbopol-940 as a viscose support bath, Lithium phenyl-2,4,6-trimethylbenzoylphosphinate (LAP) as biocompatible photo-initiator, and Quinoline yellow (QY) as photo-absorber. Once the DMD patterned UV light was projected on the reservoir, the exposed part of the prepolymer was crosslinked and converted into a solid polymer by a free polymerization reaction (FIGS. 5A, 5B, and 5C). At the end of pattern illumination, the printed construct was firmly attached to the glass coverslip. As a post-printing process, a simple washing procedure was conducted by using phosphate-buffered saline (PBS) solution, after taking out the glass slide from the reservoir to remove the non-crosslinked material. In this way, the remaining Carbopol support bath (i.e., Carbopol-940) was depolymerized, and the non-crosslinked polymer was diluted.

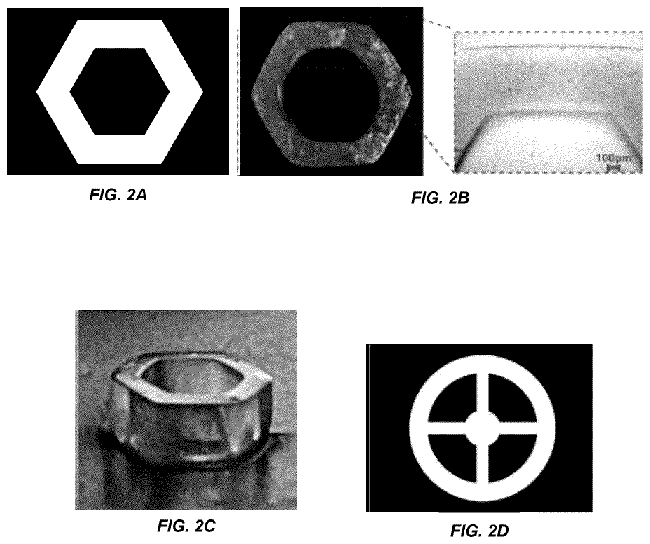

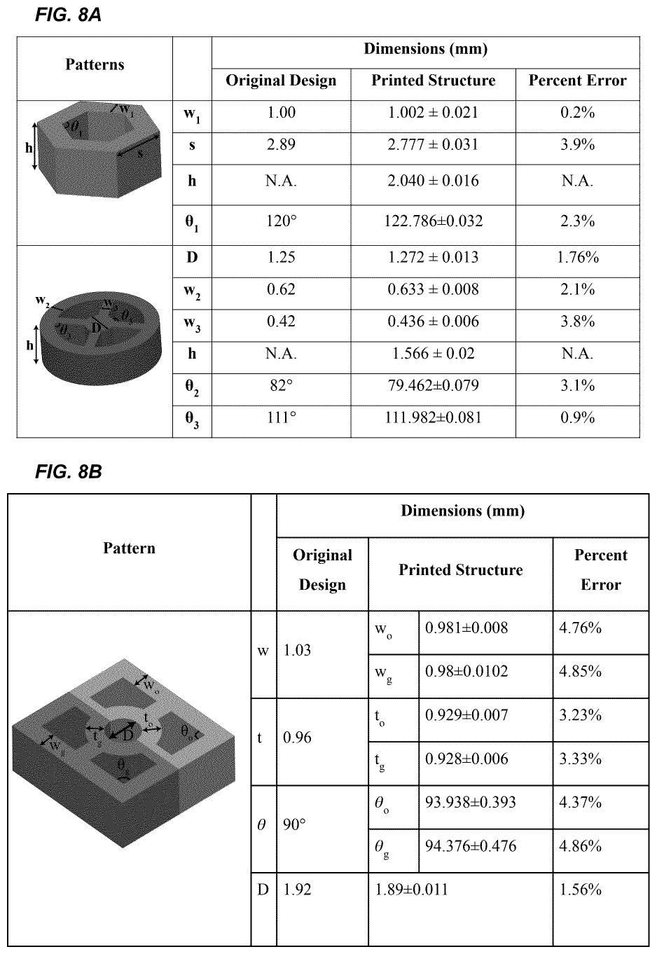

Printing of three-dimensional structures: Hexagonal ring, wheel, and cylinder structures were printed to demonstrate the printing adaptability and applicability of the POEM technique (FIG. 2A-2I). These three geometries were chosen to mimic the structure of the hepatic hexagonal lobule units, the interconnection hepatic lobule-vasculature, and the esophagus multi-layered heterogeneity, respectively. The hexagonal ring and wheel scaffolds with a single material were printed by using the two different digital masks (FIGS. 2A and 2D). Fluorescent particles were added to the prepolymer solution to obtain better visualized printed structures. The top and side views of the printed structures show that the details in the designed digital masks were successfully transferred to the printed scaffolds by single-step exposure for 3 min, as shown in FIGS. 2B-2C, and FIGS. 2E-2F. Each magnified microscope image indicated that the fluorescence particles were distributed homogeneously. Moreover, it showed that the POEM technique provided an appropriate resolution for printing the infills, and sharp inner/outer angles. The dimensions of the printed patterns were measured for comparison with the original design (FIG. 8A). For each pattern, five samples were measured to determine the printing precision of the system. There was a negligible deviation (0.2-3.9%) between the measured and designed structural parameters, meaning a precision alignment and placement. The widths of the printed structures were often higher than the designed values, due to a tiny over-curing. However, the slight differences between the measured and designed widths revealed that the proposed projection-based printing technique (i.e., POEM) has relatively acceptable accuracy and resolution.

Another point that should give attention was the thickness of the printed structures which was determined by the penetration depth of the light. The penetration depth was directed by many parameters, including the power of the light source, exposure time, the concentration of the photo-absorber, and the photo-initiator. Here, since the used exposure time, the concentration of the photo-absorber, and the photo-initiator were fixed for both structures, and their effect was ignored for the curing depth. The dominant factor was the power of the projected light. Note that the projected mask was composed of pixels, and for each pixel, the light intensity was uniformly distributed. The total power for the curing was determined by the number of ON pixels in the digital mask, in other words, the area of the pattern. For the hexagonal ring pattern, the area was around 2.1873e⁶ pixels square. For the wheel pattern, since it was composed of multiple parts, one can use the most dominant part for the evaluation. The covered area was around 6.0078e⁵ pixels square, considering the outer ring as the dominant part. Therefore, it was expected that the hexagonal ring pattern would be a thicker structure. Besides, it was not possible to state a direct relationship between the pixel number and the thickness for the given structures due to the fact that different colors of fluorescent molecules were used for them. The reason was that the light intensity was determined by the absorption and scattering characteristics of the material, so not only these parameters but also the effect of the stray beams should be considered to make the relation. Apart from that, one could manipulate the thickness of the printed structure by controlling the number of ON/OFF mirrors.

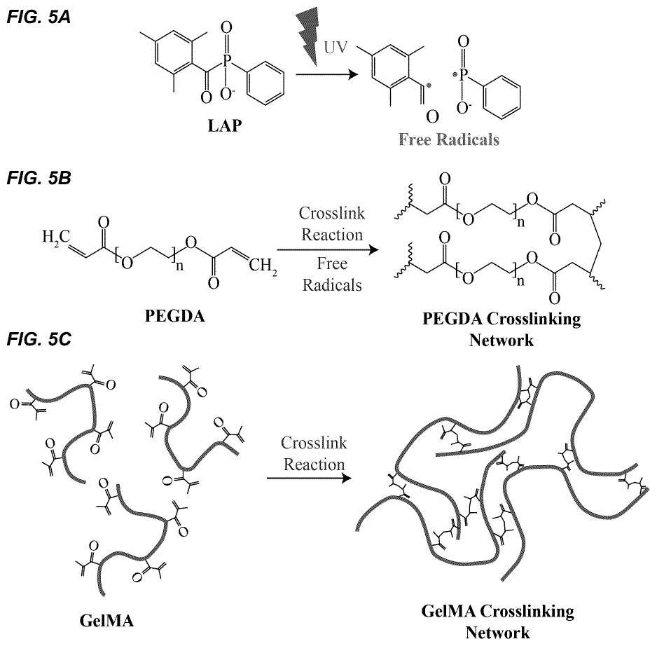

As a next step, the multi-material structures were obtained by orderly extruding the multiple prepolymer solutions into the quartz reservoir via extrusion printing and then photo-crosslinking in the DLP system. Through the POEM technique, an unrestricted number of inks/materials with multiple distribution configurations might be extruded to accommodate complex architectures. Two printing scenarios were evaluated. First, three multiple inks with colors of blue, green, and red were ordered in longitudinal configuration to assess two different geometries, cylindrical and gear shapes (FIGS. 1A, 1B, and 1C). The top view of the printed cylinder structure can be seen in FIG. 2G. Then, two inks with blue and red colors were composed to arrange the lateral layering case (FIGS. 1A, 1B, and 1C, and FIG. 2H). To confirm the color transition between the layers, fluorescent microscope images were given for both lateral and longitudinal arrangements (FIG. 2I). The influence of multi-material printing on the accuracy and resolution of the final structure was also examined by using two different materials in longitudinal layering arrangement and a mask that has vertical and horizontal symmetry, including circular, strip, and corner features, as shown in FIGS. 6A and 6B. The deviation between the measured and designed structural parameters was around 1.56-4.86% (FIG. 8B). However, the deviation between the two materials was around 0.1-0.4%, which shows that with a proper exposure time arrangement, one can get high accuracy with multi-material printing.

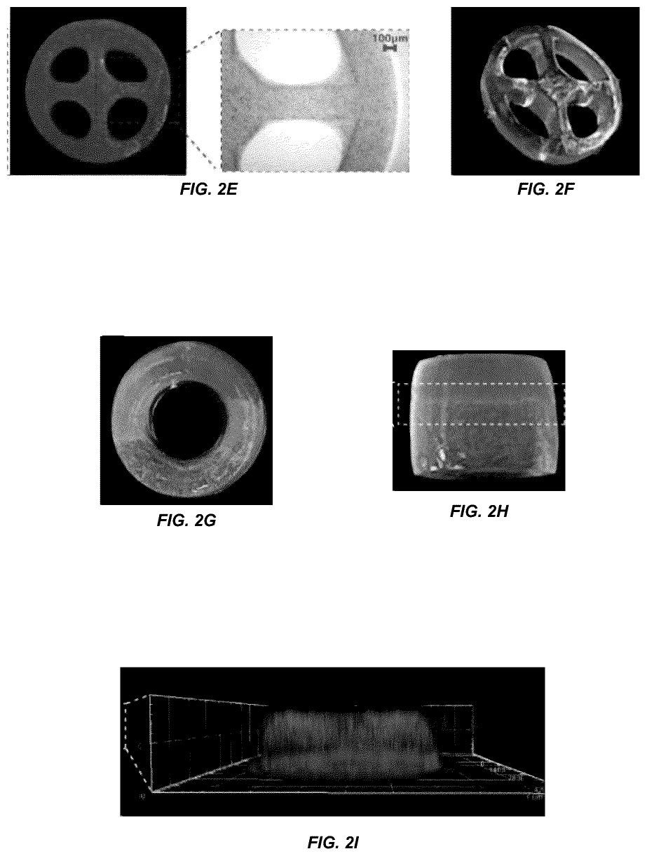

Bioprinting: The POEM technique has the potential to offer a biocompatible environment to incorporate living cells in the bioprinting process. The biocompatibility of the POEM technique, the polymers (i.e., PEGDA 700 and GelMA), and the support bath (i.e., Carbopol-940) were investigated, esophagus-liked structures were printed as a model by incorporating muscle cells (C2C12) and fibroblast cells (L929) into the PEGDA (i.e., PEGDA 700) and PEGDA+GelMA inks. The cell viability in the polymeric bioinks through the Live/Dead assay were investigated. The Live/Dead assay showed a large population of viable cells on the first day of printing 91.2±3.5% and 68.5±6.0% for PEGDA+GelMA and PEGDA, respectively (FIG. 3A). Nonetheless, a preserved cell viability of up to 80% was observed on day 5 for the PEGDA+GelMA blend. Besides, up to a 25% of reduction in cell viability was observed for PEGDA ink for 5 days (FIG. 3A). Life and dead assay confirmed significantly higher cell viability in the PEGDA+GelMA blend (***p-value≤0.001) Moreover, this tendency was confirmed by the Resazurin assay. In FIG. 3C, the bar graphs showed that the metabolic activity on day 5 decreased up to 60% in PEGDA ink, while in PEGDA+GelMA the variation was approximately 25% in comparison with metabolic activity on day 1. The reduction of cell viability in hydrogels of PEGDA might be attributed to the pore size of PEGDA which was too small to allow the medium diffusing. The pore size of PEGDA also affects the stiffness of the material and the mechanotransduction of the cells. The mechanical properties of PEGDA can be tuned by adding other photo-cross-linkable polymers like GelMA. The use of PEGDA with higher molecular weight may result in larger pores improving cell viability. As well, the formulation of photo-crosslinkable bioinks with natural-origin polymers and supplemented with additives (i.e., RGD particles, dECM, FGF, FBS) may improve the cell function and expand the bioink portfolio. The use of support baths with degradable polymers like poly(ethylene glycol) or poly (D, L-lactide) can be potentially applied in the POEM technique to generate implantable constructs.

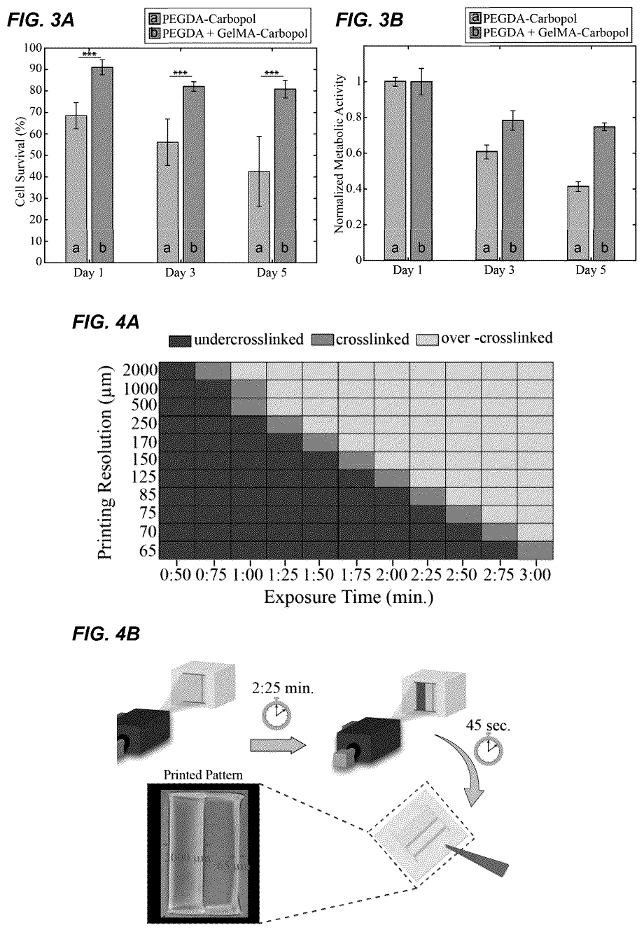

Characterization of the Photo-cross-linkable System: The performance of the POEM system was characterized by evaluating its resolution and registration capability with respect to the exposure time (0.5-3 min). To optimize the exposure time for target thickness, the PEGDA prepolymer solution was patterned, variating the exposure times for different strip thicknesses (65-2,000 μm). The minimum width of the strips was designed considering that in bioprinting, high resolution was defined as printed elements smaller than 100 μm since it was comparable to the size of a single cell. Nonetheless, the limit range of strip thickness was stated previously, since the photo-crosslinking was controlled by the pixel number, the wider thicknesses were crosslinked in a shorter exposure time (FIG. 4A). Here, the exposure time was limited to 3 min by considering the cell viability at the bioprinting part. The printing resolution featured up to 65 μm with the proposed POEM technique. Another considerable point was the engineering of the exposure time by superimposing digital masks to compound different feature sizes on a macrostructure. FIG. 4B shows the procedure of a basic case study for the superimposition of the masks. For the exposure time versus printing resolution, it was sufficient for the printing strip with 65 μm thickness with 3 min of exposure time, and 45 sec for a 2,000 μm feature size. Hence, to avoid over-crosslinking of the wider part, a mask that only contained the thinner strip was projected on the polymer. Then, the wider pattern was superimposed at the target time (i.e., 2:25 min). As shown in the presented bright field microscopy image, the strips were printed with designed thicknesses with the superimposition of the masks at proper times (FIG. 4B).

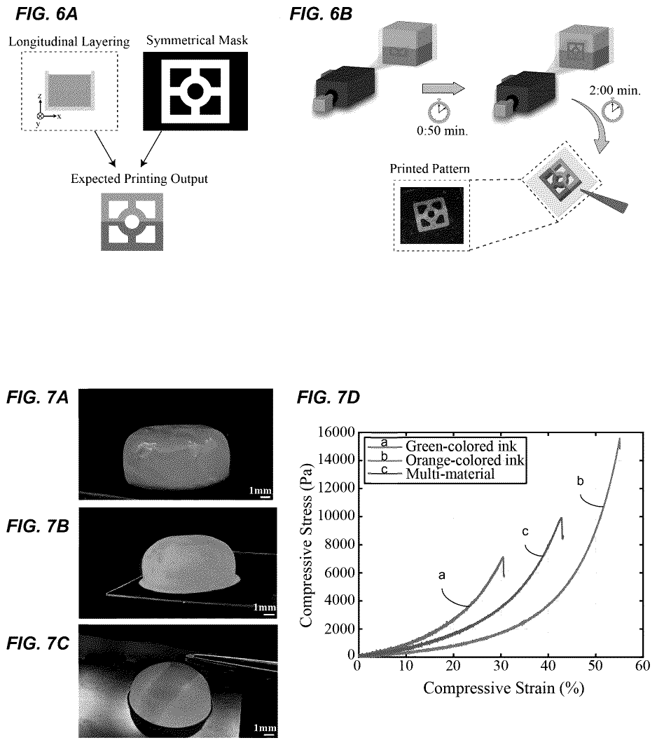

For the extrusion of inks, the rheology properties of the materials determine their feasibility to be extruded and keep the shape after the material was released from the hydraulic pressure. A rheology characterization of all the materials was conducted; Carbopol, PEGDA prepolymer, PEGDA-Carbopol, PEGDA+GelMA, and PEGDA+GelMA-Carbopol (FIG. 4C). All the materials showed a shear-thinning behavior, meaning that their viscosity decreases with increasing shear rates. This confirms their capability to be implemented in 3D-extrusion-printing. The viscosities of the materials evaluated at the lowest shear rate were 0.91±0.13, 1.20±0.02, 21.66±2.97, 51.36±1.60, and 31.47±2.51 Pa's for PEGDA prepolymer, PEGDA+GelMA, PEGDA-Carbopol, Carbopol, and PEGDA+GelMA-Carbopol, respectively (FIG. 4C). As expected, the incorporation of the Carbopol support bath to PEGDA, the PEGDA+GelMA yields inks with higher viscosity while preserving the shear thinning behavior. The absorbance spectra of all materials were investigated (FIG. 4D). Firstly, the absorbance of Carbopol presented minimal interference with the light, due to its transparency and the absence of any impurity. Secondly, QY absorbs in the range between 360-475 nm, with its higher absorbance at 410 nm. The light absorption of QY has attenuated in combination with PEGDA, PEGDA+GelMA, and Carbopol, due to the dissolution of concentration. The swelling properties of a polymer network reflect its mechanical properties, diffusion capability, and surface mobility. The swelling behavior of PEGDA-Carbopol and PEGDA+GelMA-Carbopol xerogels showed a rapid swelling in the first 5 h achieving stable equilibrium in about 16 h. The water swelling increased with time, and the constructs increased their swelling ratio by ~1,500% compared to their dry weight due to the water retention of PEGDA. This swelling behavior was similar to the one reported by other authors. The data demonstrated the ability of GelMA to reduce the swelling ratio in comparison with PEGDA-Carbopol. The mechanical properties of hydrogels affected cell proliferation and differentiation. Thus, it was important to measure the mechanical properties of the PEGDA-Carbopol and the PEGDA+GelMA-Carbopol. The compressive strength of the PEGDA-Carbopol (~139.9 Pa) results was higher than PEGDA+GelMA-Carbopol (~35.6 Pa), due to the rigidity of PEGDA chains. To elucidate the changes in mechanical properties of single materials while applied to fabricate multi-material structures, hemispheres were printed and tested under compression test (FIG. 7A-7D), assuming that if there was uniform crosslinking for the multi-material printing, the mechanical properties of the multi-material structure should show stiffness characteristics which was the average of the materials that compose it. Uniaxial compression showed differences in the mechanical properties of green- and orange-colored inks. Specifically, the average onset of fracture of green- and orange-colored inks were ~32 and ~55%, respectively. In contrast, the multi-material constructs displayed fracture points in compressive strains of ~43% between their individual components. To emulate the tissue mechanical properties, hydrogels might be tuned with the co-formulation of small molecular weight PEGDA and high molecular weight hydrogels like GelMA.

Resolution Analysis for Multi-material Printing: The light-matter interaction of each material differs from each other; hence for the multi-material case to be able to get desired accuracy and resolution, the exposure time and the local power intensity coming from the DMD mirrors should be engineered. Here a case study is presented to examine the influence of multi-material printing on accuracy and resolution. For that purpose, two different materials in longitudinal layering arrangement and a mask that has vertical and horizontal symmetry, including circular, strip, and corner features (FIG. 6A) were used.

Before starting multi-material printing, the crosslinking property of each material should be analyzed. Depending on the analysis, the green colored ink needs 0:50 min. more light exposure when compared with the orange colored one to get desired structural parameters. Thus, the superimposition of the masks approach was used. As shown in FIG. 6B, first the half of the mask was projected on the green material for 0:50 minutes and then the whole mask projected on both materials for 2:00 minutes. The dimensions of the printed patterns were measured for comparison with the original design. To see the effect of the multi-material printing of FIG. 8B was prepared by showing the dimensions separately for orange/green material and then for the whole structure. The deviation between the measured and designed structural parameters were around 1.56-4.86%. However, the deviation between two materials was around 0.1-0.4%, which shows that with a proper exposure time arrangement one can get high accuracy with multi-material printing.

Photocuring Uniformity Test: To test the photocuring uniformity, first, two different PEGDA base inks, green- and red-colored were prepared. Then, hemisphere structures were printed with single-material (green/red-colored) and multi-material configurations FIG. 7A-7D.

The side views of the structures are presented in FIG. 7A-7C. The compression modulus of individual single- and multi-material constructs to see how stiffness of the printed constructs was affected by the multi-material configuration were evaluated. To assess the mechanical properties, the height and the diameter of the printed hemispheres structures were measured with a Vernier caliper. Then the hemispheres were loaded on the universal Testing System (Instron 3365 UTS) and compressed unconfined at a compression rate of 200 μm/min. The compression test was replicated 5 times, and the representative curves were plotted as shown in FIG. 7D. Noting that the stiffness of the construct depends on the ink composition and the photo-crosslinking process, the multi-material structures possess different physicochemical characteristics than those which compose it individually. Uniaxial compression showed differences in the mechanical properties of green- and orange-colored inks. Specifically, the average onset of fracture of green- and orange-colored inks were ~32 and ~55%, respectively. In contrast, the multi-material constructs displayed fracture points in compressive strains of ~43% between their individual components. The modulation of the mechanical properties was also possible by varying the composition of the ink formulations and the exposure time.

State-of-the-art DLP techniques bear on complex imaging processing, require highly skilled personnel, operate with non-biocompatible/photo-cross-linkable materials and were not yet capable of multi-layer and multi-material printing of biocompatible/photo-cross-linkable materials to fabricate physiologically relevant cell-laden structures. Herein, a novel DLP-based 3D-bioprinting technology has been developed, and fully characterized. The POEM technique principle of working was extrusion bioprinting of photo-cross-linkable hydrogels in a layer-by-layer manner followed by high-resolution patterning of the layers to the desired shapes and configurations using a 4-f lens system. The utility of the POEM technique for rapid and high-resolution 3D-printing of multi-material, multi-layer, and cell-laden structures was demonstrated. The printed configurations showed high cell viability (~80%) and metabolic activity for more than five days. As a study model, a 3D structure representing the esophagus was also successfully printed and characterized. The POEM technique described herein maintains a high printing resolution, offers multi-material printing to achieve the heterogeneity presented in actual tissues and multicellular structures, eliminates cross-contamination and the cleaning process that was required in other state-of-the-art multi-material printing techniques, and enables multilayer printing to structurally mimic the in vivo tissue architectures. Moreover, the use of a support bath implemented in the POEM technique holds the 3D-bioprinted structures still during the entire process and hence eliminates the chance of collision and any structural deformations.

Materials: Poly (ethylene glycol) diacrylate (PEGDA-700) Mn 700 and Quinoline Yellow (QY) were purchased from Sigma Aldrich, USA. Carbopol-940 was purchased from Acros Organics. Lithium phenyl-2,4,6-trimethyl-benzoyl phosphinate (LAP) was purchased from Allevi. DMEM media, ANTI-ANTI, and Fetal Bovine Serum were purchased from Gibco. The UV-395 nm torch was purchased from Darkbeam-Amazon. DLP Projector (DLP LightCommander) was purchased from Texas Instruments, USA. Lenses, iris, and mirrors were purchased from Thorlabs.

GelMA synthesis: GelMA was synthesized by dissolving type A gelatin from porcine skin at 10% (w/v) in Dulbecco's phosphate-buffered saline (DPBS) and stirring at 50° C. and 300 rpm for an hour. Methacrylic acid was added dropwise with a syringe pump (0.3 mL/min) to the solution at 5% (v/v) and allowed to react for 1 h. The reaction was finished by adding 5× volumes of DPBS. The resulting solution was dialyzed for 7 days with distilled water (DW) at 37° C. The resulting solution was frozen and lyophilized for five days and stored at −80° C. before use.

Preparation of PEGDA Ink: To prepare the prepolymer PEGDA inks, 20% w/v solution of PEGDA-700 was mixed with 0.1% w/v QY. Then, the PEGDA solution was heated at 65° C. for 20 min. This process was followed by the addition of 0.5% w/v LAP (ALEVY) photo-initiator and pH neutralization with NaOH (1 M). The PEGDA solutions were sterilized using 0.22 μm polyether sulfone (PES) filters, before being applied for 3D-bioprinting of the cells.

Preparation of GelMA ink: Lyophilized GelMA was dissolved at 10% (w/v) in DW and mixed with 0.025% w/v QY. Followed by the addition of 0.1% (w/v) of LAP as the photo-initiator and pH neutralization with NaOH (1 M). Finally, GelMA solutions were sterilized using 0.22 μm PES filters.

Preparation of Carbopol Support Bath: The synthesis of the Carbopol support bath was adapted to the POEM technique. Briefly, a 1.2% w/v solution of Carbopol-940 was prepared by vortex mixing in DW, then 0.5 mL of NaOH (1 M) was added. After that, the Carbopol support bath was mixed and centrifuged at 3,000 rpm for 20 min.

Preparation of PEGDA-Support bath: The PEGDA-Carbopol support bath was prepared by mixing 3 mL of PEGDA ink in 7.5 mL of the Carbopol-940 support bath.

Preparation of PEGDA+GElMA-Support bath: The PEGDA+GelMA-Carbopol-940 support bath was prepared by mixing 1 mL of PEGDA ink with 2 mL of GelMA in 7.5 mL of the Carbopol-940 support bath.

Rheological Test: The rheological properties of prepolymer PEGDA ink, Carbopol support bath, and the mix of PEGDA-Carbopol were investigated by using a DHR rheometer (TA Instruments) equipped with a Peltier. A 20 mm parallel plate with a 200 μm gap was used for all rheological tests. PEGDA, Carbopol-940, and PEGDA-Carbopol mix were loaded, and their complex viscosity was studied as a function of shear rate (1-100 Hz) at 24° C. with a constant strain of 2% (n=3).

Absorption spectra of the materials: Absorption spectra of the prepolymer PEGDA ink, Carbopol support bath, and the mix of PEGDA-Carbopol were measured by a UV-Visible spectrophotometer Biotek Cytation 5/Biospa Plate Reader, and the results were plotted as a function of wavelength in a range from 200-800 nm. Polymers were prepared as described previously, and a blank of DW was used.

Compressive modulus: To test the mechanical properties, 5 ml of PEGDA-Carbopol solution was extruded into a 12-well plate and crosslinked at 395 nm with UV light for 60 sec. Then the samples were taken off the well plate. Young's modulus was tested by a dynamic mechanical analysis instrument (Instron 3365 UTS). The compressive modulus was determined as the slope of the linear region between strains from 5 to 20%.

Hydrogel swelling: For the measurement of water swelling, six cylindrical samples were prepared using the protocol described above. To leach the unreacted-soluble fraction, the samples were placed in water at room temperature for 24 h. Then samples were dried in a vacuum desiccator for 2 days. Next, the dried samples were immersed in DW at 37° C. The swelling kinetics was studied after taking out the soaked samples from water at different time points and weighing them once the surface droplets were wiped off with wet paper until getting constant weight. The swelling ratio was determined by the following equation:

$${S}_{w} ( \% ) = \frac{{W}_{t} - {W}_{0}}{{W}_{0}} \times 100 \qquad ( 1 )$$

where Wₜ was the weight of the hydrogel sample at a specific time, and W₀ was the weight of the dried samples recorded as the initial weight. All experiments were performed in triplicate.

DLP Printing: Printing was performed by customizing the projection lens and illumination source of the commercial DLP (Texas Instruments, Dallas, TX). Instead of using its illumination module with a visible light source, a UV source operating at 395 nm was integrated for the illumination of the DMD (Texas Instruments, Dallas, TX) chips. The projection optics was replaced with a 4-f system, and the optical path was adjusted with an ×0.7 scaling by properly arranging the focal lengths of the lenses. The DMD chip in the system consists of 1,024×768 individually controllable micromirrors of 10.8 μm size. Hence, one pixel on a mask reflected by one mirror on the DMD chip was about 7.56×7.56 μm2 after being projected from the 4-f system to the target pre-polymer. All digital masks were designed in Adobe Illustrator and loaded as BMP files into the projection software of the DLP printer.

A quartz reservoir was filled with the polymer+support bath (which functions as a holder for the printed structure) mixture, and a glass slide (size: 18×18 mm) was placed in between the reservoir wall and the polymer to easily remove the printed pattern without damaging it. Then, the quartz reservoir was placed over the stage and the mask was projected against the reservoir wall.

Photopolymerization of orderly extruded multi-material: Firstly, the polymers+support bath was transferred into cartridges and degassed by centrifugation at 2,000 rpm for 10 min; subsequently, cartridges were loaded in the 3D-extrusion-bioprinter (Incredible+, Cellink, Sweden). Then the G-code to fill the reservoir in the desired pattern was uploaded. The materials were orderly extruded inside the quartz reservoir using 14 G needles, and the final product was covered with parafilm.

Printing resolution: To evaluate the printing resolution of the system, a calibration structure consisting of an array of strip features of decreasing size and gaps was printed. After recovering the samples, the fidelity of printed structures was evaluated by using image analysis. Photographic images of printed structures were analyzed using Toupview software (AmScope, USA) and compared to the original design parameters (n=3).

Cell culture: Mouse fibroblast L929 was kindly provided by Professor Masashi Kitazawa from the UC Irvine School of Medicine, and mouse myoblast C2C12 kindly provided by Professor Angela G. Fleischman from UCI School of Medicine was used for this study. Bout cell lines were cultured in an incubator at 37° C. under a 5% CO2 atmosphere. The standard cell culture media was made from Dulbecco's Modified Eagle Medium (DMEM) supplemented with 10% of fetal bovine serum and 1% of Anti-Anti.

Cell viability assay: Cell viability test of L929 and C212 was proceeded on days 1, 3, and 5 by performing a LIVE/DEAD assay (Life Technologies, California, United States) which contained 2 mM calcein-AM and 4 mM ethidium homodimer (EtHD1). The samples were placed in a dark humidifier incubator for 30 min. Then constructs were washed with PBS and immediately imaged using The Zeiss LSM 900 Airyscan 2 confocal fluorescence microscope (Zeiss, Germany).

Metabolic activity analyzes: Metabolic activity was accessed on days 1, 3, and 5 using the Reassuring assay (Biotium, USA). Briefly, constructs were covered in DMEM culture media with 10% v/v Reassuring reagent and incubated for 2 h at 37° C. After incubation in a 96-well plate, 100 μL of media were transferred, and the fluorescence was measured in a microplate reader at 530/571 nm excitation/emission wavelengths. Fluorescence readings were normalized with respect to control well with the medium in the absence of biological samples.

Statistical analysis: Analysis of variance was performed with SPSS 27 (IBM, USA). Differences with a p-value <0.001 (***) were considered statistically significant.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from a 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java. Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method for three-dimensional (3D) bioprinting of multi-layer multi-material structures, the method comprising:
a) preparing a plurality of prepolymers and a plurality of living cells incorporated into at least one of the plurality of prepolymers;
wherein the plurality of prepolymers comprises a plurality of prepolymer types;
wherein the plurality of living cells comprises a plurality of cell types;
b) applying a thickening agent to the plurality of prepolymers; and
c) patterning the plurality of prepolymers into a three-dimensional shape comprising one or more layers such that adjacent prepolymers differ in prepolymer type, cell type of one or more living cells incorporated into each prepolymer, or a combination thereof, wherein patterning comprises photo-cross-linking the plurality of prepolymers to form a hydrogel, wherein the plurality of living cells are encapsulated in the hydrogel.

2. The method of claim 1, wherein the plurality of prepolymer types comprise poly(ethylene glycol) diacrylate (PEGDA), gelatin methacryloyl (GelMA), alginate methacrylate (ALMA), silk methacrylate (SilMA), collagen methacrylate (ColMA), methacrylated poly(vinyl alcohol) (PVA-MA), methacryloyl-substituted recombinant human tropoelastin (MeTro), any decellularized extracellular matrix-methacrylate (dECM-MA), or a combination thereof.

3. The method of claim 1, wherein preparing the plurality of prepolymers comprises putting the plurality of prepolymers into a printer cartridge and extruding the plurality of prepolymers into a reservoir.

4. The method of claim 3, wherein the plurality of prepolymers are extruded into a hexagonal ring, a wheel, or a cylinder structure.

5. The method of claim 1, wherein the one or more layers of prepolymer comprise distinct boundaries between the one or more layers of prepolymer.

6. The method of claim 1, wherein a digital light processing (DLP)-printing system is used for patterning the plurality of prepolymers.

7. The method of claim 6, wherein the DLP-print system comprises:
a) an ultraviolet (UV) light source configured to generate UV light;
b) a computing device comprising a processor configured to execute computer-readable instructions and a memory component operatively coupled to the processor, comprising computer-readable instructions for generating, adjusting, and sending a digital mask sequence;
c) a digital micromirror device (DMD) chipset communicatively coupled to the computing device, disposed optically in-line with the UV light source, configured to receive the digital mask sequence and modulate the UV light based on the digital mask sequence to form a digital mask image;
d) an optical projection lens system disposed optically in-line with the DMD chipset, configured to project the digital mask image; and
e) a stage for sample positioning disposed optically in-line with the optical projection lens system, wherein the digital mask image is projected onto the stage.

8. The method of claim 7, wherein the optical projection lens system is further configured to filter the digital mask image at a Fourier plane.

9. The method of claim 7, wherein the DMD chipset comprises a plurality of mirrors, wherein the memory component further comprises instructions for controlling an operation time of each mirror of the plurality of mirrors of the DMD chipset.

10. The method of claim 1, further comprising post-processing of the hydrogel.

11. The method of claim 10, wherein post-processing of the hydrogel comprises washing the hydrogel to remove non-crosslinked prepolymer.

12. The method of claim 11, wherein the hydrogel is washed with a phosphate-buffered saline (PBS) solution.

13. A multi-layer, multi-material, three-dimensional (3D) printed article comprising a hydrogel and living cells, wherein the hydrogel is formed from a pattern of prepolymers comprising a thickening agent, wherein the living cells are incorporated into the pattern of prepolymers, wherein adjacent prepolymers in the pattern of prepolymers differ in prepolymer type, cell type, or a combination thereof.

14. The 3D printed article of claim 13, wherein the article is shaped into a hexagonal ring, a wheel, or a cylinder structure.

15. A three-dimensional (3D) printer cartridge containing a printing solution comprising a plurality of prepolymers having a plurality of prepolymer types, a plurality of living cells having a plurality of cell types, a thickening agent applied to the plurality of prepolymers, and a photo-initiator.

16. The 3D printer cartridge of claim 15, wherein the photo-initiator is a biocompatible photo-initiator.

17. The 3D cartridge of claim 15, wherein the photo-initiator is lithium phenyl-2,4,6-trimethylbenzoylphosphinate (LAP), 2-Hydroxy-4′-(2-hydroxyethoxy)-2-methylpropiophenone (Irgacure 2959), Eosin Y, VA086, or a combination thereof.

18. The 3D cartridge of claim 15, wherein the plurality of cell types comprise endothelial cells, fibroblast cells, epithelial cells, muscle cells, bone cells, cartilage cells, heart muscle cells, liver cells, neurons, fat cells, keratinocytes, cancerous cells, stem cells, macrophages, or a combination thereof.

19. The 3D cartridge of claim 15, wherein the plurality of prepolymer types comprise poly(ethylene glycol) diacrylate (PEGDA), gelatin methacryloyl (GelMA), alginate methacrylate (ALMA), silk methacrylate (SilMA), collagen methacrylate (ColMA), methacrylated poly(vinyl alcohol) (PVA-MA), methacryloyl-substituted recombinant human tropoelastin (MeTro), any decellularized extracellular matrix-methacrylate (dECM-MA), or a combination thereof.

20. The 3D cartridge of claim 15, wherein the thickening agent comprises Carbopol-940, gelatin, alginate, or a combination thereof.
